(19) United States
(12) Patent Application Publication
(10) Pub. No.: US 2019/0262541 A1
(43) Pub. Date: Aug. 29, 2019

(54) IMAGE CAPTURE DEVICE WITH VIEWING FUNCTIONALITY

(71) Applicant: Novo Nordisk A/S, Bagsvaerd (DK)

(72) Inventor: Martin Johst Christensen, Copenhagen OE (DK)

(21) Appl. No.: 16/343,456

(22) PCT Filed: Oct. 30, 2017
(86) PCT No.: PCT/EP2017/077788

§ 371 Date: Apr. 19, 2019

(30) Foreign Application Priority Data
Oct. 31, 2016 (EP) 16196559.5

(51) Int. Cl. A61M 5/315; H04N 5/225; G06K 9/00; G06T 7/20; G02B 27/10; G02B 5/20; G02B 27/14

(52) U.S. Cl. CPC A61M 5/3157; A61M 2005/3126; A61M 5/31541; H04N 5/2252; H04N 5/2256; G06K 9/00624; H04N 5/2253; G06T 7/20; G02B 27/106; G02B 5/208; G02B 27/141; A61M 2205/3306; A61M 2205/50; A61M 2205/52; A61M 2205/3313; A61M 2205/583; A61M 5/31568

(57) ABSTRACT

An image capture device with a housing comprising a capture opening and a viewing opening arranged opposed to the capture opening. The image capture device comprises a camera for capturing an image of at least a portion of an object arranged in register with the capture opening, as well as a beam-splitter arranged to cover the capture opening, the beam-splitter allowing a first portion of light reflected from an observed object to pass through the beam-splitter, this allowing a user to effectively watch the object during image capture.

The present invention generally relates to arrangements allowing an image to be both captured and observed. In a specific aspect the invention relates to medical devices and assemblies for which the generation, collecting and storing of data are relevant. In specific embodiments the invention relates to devices and systems for capturing drug delivery dose data in a reliable, user-friendly and power efficient way.

### BACKGROUND OF THE INVENTION

In the disclosure of the present invention reference is mostly made to drug delivery devices comprising a threaded piston rod driven by a rotating drive member, such devices being used e.g. in the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device it-self. Addressing this problem a number of solutions have been proposed which would help a user to generate, collect and distribute data indicative of the use of a given medical device.

For example, WO 2013/120776 describes an electronic supplementary device (or “add-on module”) adapted to be releasably attached to a drug delivery device of the pen type to determine and store expelled dose amounts. The device includes a camera and is configured to perform optical character recognition (OCR) on captured images from a rotating scale drum visible through a dosage window on the drug delivery device, thereby to determine a dose of medicament that has been dialled into the drug delivery device. As the device covers the dosage window the camera is also used to capture data during dose setting, this allowing the current scale drum value to be shown on an electronically controlled display during dose setting. A further external add-on device for a pen device is shown in WO 2013/004843, this device comprising a camera arrangement allowing the user to observe the scale drum during dose setting. WO 2013/120778 discloses a monitoring device in which dose size determination is based on sensors adapted to detect axial and rotational movement.

Having regard to the above, it is an object of the present invention to provide devices, assemblies and methods allowing an object to be both captured and observed in an effective way. It is a further object to provide devices, assemblies and methods allowing secure, easy and cost-effective operation of a drug delivery assembly comprising a user-mountable add-on module.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first general aspect of the invention an image capture device comprising a housing (301) comprising with a first surface, a capture opening formed in the first surface, and a viewing opening arranged opposed to the capture opening. The viewing opening will typically be provided with a transparent member or formed by a transparent portion of the housing.

The image capture device further comprises a memory, capturing means adapted to capture an image of at least a portion of an object arranged in register with the capture opening, the capturing means being arranged off-set in respect of the viewing axis between the viewing opening and the capture opening, and a processor adapted to process captured images and store data in the memory. The capture device further comprises a beam-splitter device arranged to cover at least a portion of the capture opening viewable through the viewing opening, the beam-splitter device being adapted to allow a first portion of light reflected from an object arranged in register with the capture opening to pass through the beam-splitter device, this allowing a user to watch a portion of the object covered by the beam-splitter device, and allow a second portion of light reflected from the indicator member to be reflected by the beam-splitter device towards the capturing means.

By this arrangement an image capture device is provided which effectively allows an object to be both viewed by a person an captured by electronic capturing means for subsequent storage and/or processing.

The capturing means may comprise an image sensor just as it may further comprise a mirror arranged to direct light received from the beam-splitter device towards the capturing means, this allowing a greater freedom of design when arranging the components of the image capture device in the housing. Alternatively, the light reflected from the beam splitter may be directed directly towards the capturing means.

In an exemplary embodiment the image capture device further comprises a light source arranged to illuminate at least a portion of an object arranged in the vicinity of the capture opening and viewable through the viewing opening, the capturing means comprising a light filter adapted to filter out at least a portion of light outside the range of light from the light source. The light source may be in the form of an IR light source and the light filter may be adapted to filter out at least a portion of light outside the IR range. The beam-splitter device may be of the dichroic type and may be adapted to primarily reflect IR light and transmit visible light.

Alternatively the image sensor may be adapted to operate using ambient externally supplied natural or artificial light, e.g. the device housing may have window portions allowing the observed object to be illuminated from the outside.

In a further exemplary embodiment the processor is adapted to recognize a numerical value in a captured image, and determine and store in the memory a calculated numeric value representing the difference between first and second recognized numerical values. The images may be captured from a scale drum of a drug delivery device, the observed portion of the scale drum comprising indicia representing a numerical value for a presently set dose amount.

In a further aspect of the invention an image capture device of the above-described type is configured to be releasably attached to a drug delivery device, the drug delivery device comprising a drug reservoir or a compartment for receiving a drug reservoir, drug expelling means comprising a dose setting member allowing a user to set a dose amount of drug to be expelled, an indicator member adapted to rotate relative to the housing during dose setting and dose expelling corresponding to an axis of rotation, the amount of rotation corresponding to a set dose respectively the amount of drug remaining to be expelled from a reservoir by the expelling means, the indicator member having an initial rotational position corresponding to no dose amount being set, a housing comprising an opening allowing a user to observe a portion of the indicator member, and a pattern arranged circumferentially or helically on the indicator member, comprising a plurality of indicia, the currently observable indicia indicating to a user the size of a currently set dose amount of drug to be expelled. The image capture device is adapted to determine, when mounted to a drug delivery device housing, an expelled dose amount, the image capture device comprising mounting means adapted to releasably mount the image capture device to the drug delivery device in a predetermined position and orientation with the first surface facing the drug delivery housing and the capture opening arranged in register with the housing opening. The capturing means is adapted to capture an image of at least a portion of a currently observable indicia on the indicator member, and the processor is adapted to determine and store an expelled dose-amount based on first and second captured images of the indicator member, the first image being captured when a dose amount has been set and the second image being captured when a dose amount has been expelled.

Providing a “virtual” display for an “add-on” accessory device which covers the scale drum window during operation results in a higher degree of complexity just as the required additional display adds to the size and cost of the device. Further, although it may be possible to provide a virtual display which is accurate and reliable to a very high degree, users may be concerned that this is not always the case. Correspondingly, by the above arrangement an add-on device for a drug delivery device is provided which allows data to be captured from a displayed scale drum value yet allows the user to operate and use the drug delivery in the normal way, i.e. setting a dose of drug to be expelled using the scale drum values directly.

The add-on image capture device may further comprise motion sensor means adapted to detect that a dose is being set. In an exemplary embodiment the motion sensor means is adapted to detect motion based on images captured by the capturing means and thereby motion of the indicator member. Alternatively, the motion sensor means may be adapted to detect motion of the dose setting member. For example, the image capture device may comprise a user-operated additional dose setting member arranged to engage a dose setting member of the drug delivery device, the dose setting member forming part of a motion sensor arrangement. The motion sensor may be based on a mechanical, optical, magnetic or conductive interface with the moving member.

Detected motion information may be used by the processor to more efficiently determine expelled dose amounts, e.g. by supplying motion and position data which will aid in processing the information contained in the captured images.

The image capture device may further comprise a release member actuatable between a dose setting state and an expelling state in which the drug expelling means is released to expel a set dose, as well as state sensor means adapted to detect the state of the release member. For example, the image capture device may comprise a user-operated additional dose release member arranged to engage a release member of the drug delivery device, the add-on release member forming part of a state sensor arrangement. The state sensor may be based on a mechanical, optical, magnetic or conductive interface with the moving member.

In an exemplary embodiment the image capture device comprises an electronically controlled display adapted to display data related to determined dose amounts, e.g. the size and the corresponding time for a stored dose events. Alternatively or in addition, the image capture device may comprise communication means allowing data related to determined dose amounts to be transferred to an external device, e.g. wireless transmission means such as NFC or Bluetooth® allowing data to be transferred to and displayed on a smartphone.

A mechanical mounting switch may comprise a switch member protruding from a mounting surface and being adapted to engage the drug delivery device.

In a specific embodiment an image capture device is provided in the form of an add-on device configured to be releasably attached to a drug delivery device is provided, the drug delivery device comprising a drug reservoir or a compartment for receiving a drug reservoir, drug expelling means comprising a dose setting member allowing a user to set a dose amount of drug to be expelled, an indicator member adapted to rotate relative to the housing during dose setting and dose expelling corresponding to an axis of rotation, the amount of rotation corresponding to a set dose respectively the amount of drug remaining to be expelled from a reservoir by the expelling means, the indicator member having an initial rotational position corresponding to no dose amount being set, a housing comprising an opening allowing a user to observe a portion of the indicator member, and a pattern arranged circumferentially or helically on the indicator member, comprising a plurality of indicia, the currently observable indicia indicating to a user the size of a currently set dose amount of drug to be expelled. The add-on device is adapted to determine, when mounted to a drug delivery device housing, an expelled dose amount. The add-on device comprises mounting means adapted to releasably mount the add-on device to the drug delivery device in a predetermined position and orientation, a memory, capturing means adapted to capture an image of at least a portion of a currently observable indicia, and a processor adapted to determine and store an expelled dose amount based on first and second captured images of the indicator member, the first image being captured when a dose amount has been set and the second image being captured when a dose amount has been expelled. The add-on device further comprises a beam-splitter device arranged to cover at least a portion of the indicator member viewable through the housing opening when the add-on device is attached to a drug delivery device. The beam-splitter device is adapted to allow a first portion of light reflected from the indicator member to pass through the beam-splitter device, this allowing a user to watch a portion of the indicator member covered by the beam-splitter device, and allow a second portion of light reflected from the indicator member to be reflected by the beam-splitter device towards the capturing means.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as noninsulins such as GLP-1 and analogues thereof. In the description of exemplary embodiments reference will be made to the use of insulin, however, the described module could also be used to create logs for other types of drug, e.g. growth hormone.

### BRIEF DESCRIPTION OF THE DRAWINGS

In the following embodiments of the invention will be described with reference to the drawings, wherein

FIG. 1A shows a pen-formed drug delivery device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIG. 4 shows a schematic representation of an add-on device with a display mounted on the housing of a drug delivery device,

FIG. 5 shows a schematic representation of an add-on image capture device without a display mounted on the housing of a drug delivery device,

FIG. 6 shows in part a further add-on device mounted on the housing of a drug delivery device,

FIG. 7 shows a scale drum reference representation,

FIG. 8 shows an image capture from a scale drum,

FIG. 9 shows cross correlation of the FIG. 8 image portion to the reference representation, and

FIG. 10 shows a matched portion of the reference representation.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

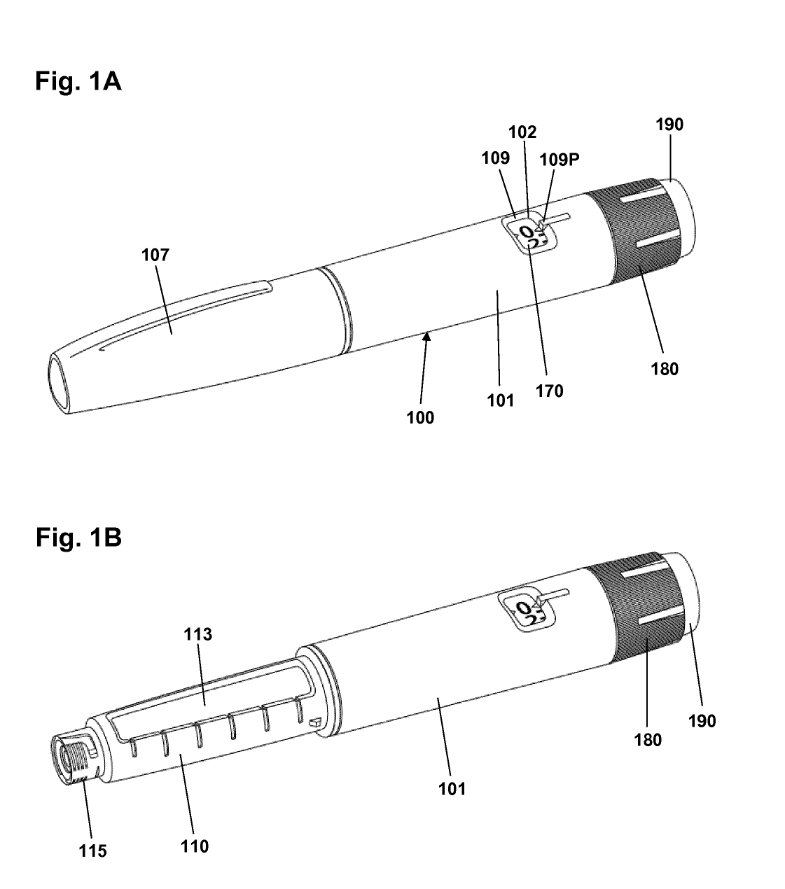

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsvrd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 serves to manually set a desired dose of drug shown in display window 102 and which can then be expelled when the button 190 is actuated. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button moves proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

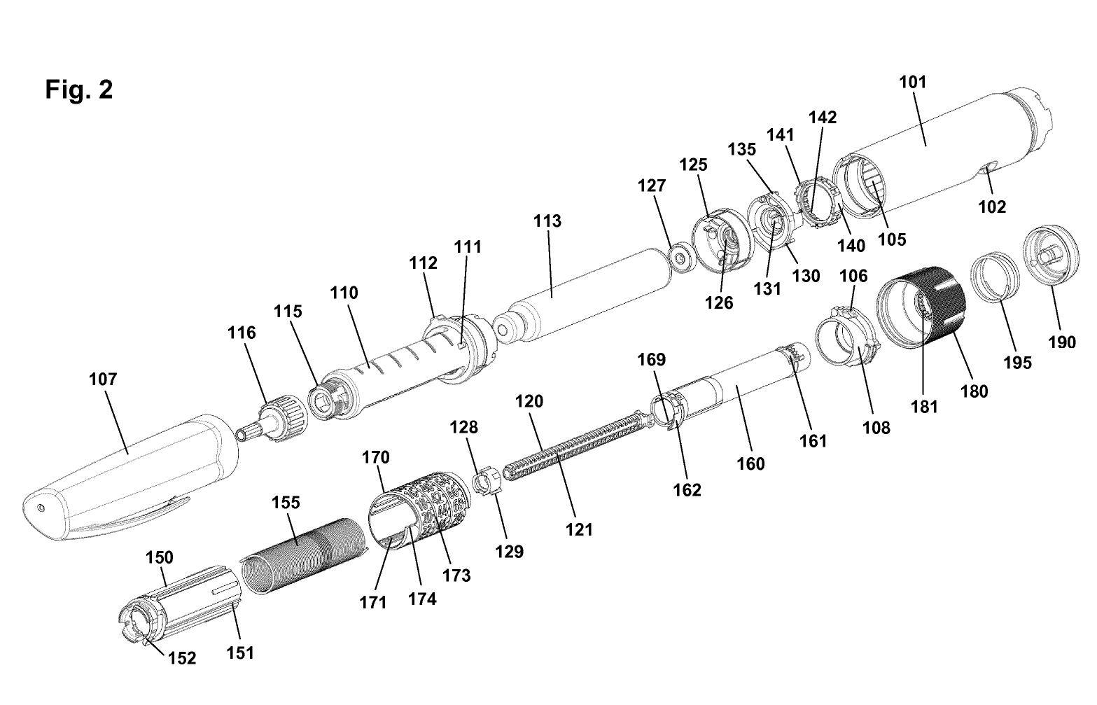

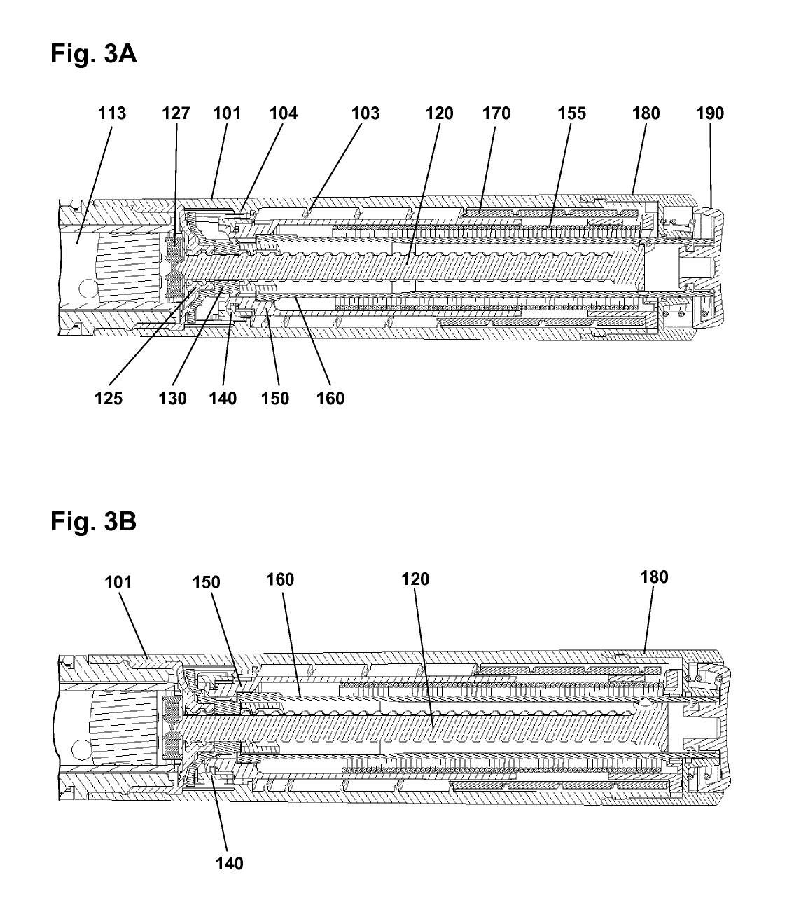

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a table top. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of dial ring results in a corresponding rotation of the reset tube and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system, the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled.

Turning to the dial system, the dose is set and reset by turning the dial member 180. When turning the dial, the reset tube 160, the EOC member 128, the ratchet tube 150 and the scale drum 170 all turn with it. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A dose of less than 5 IU normally cannot be paused, since the rubber piston is compressed very quickly leading to a compression of the rubber piston and subsequently delivery of insulin when the piston returns to the original dimensions.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialing mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “over dose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click arm 106 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with a circumferential inner teeth structure 181 engaging a number of corresponding teeth arranged on a flexible carrier portion 161 of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Before turning to the present invention per se, an example of an add-on device having a design in which the user cannot observe the scale drum window during dose setting will be described, the device instead being provided with a display adapted to “virtually” show the currently dialled dose size.

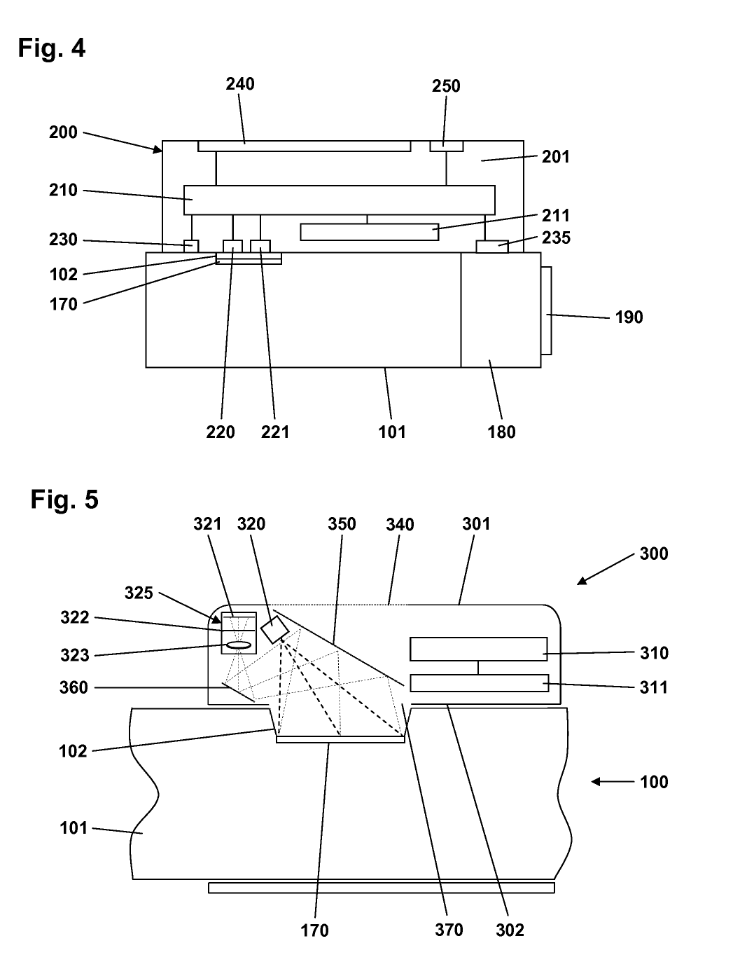

More specifically, FIG. 4 shows a schematic representation of an add-on device (or module) 200 in a state where it has been mounted on the housing 101 of a drug delivery device 100 of the above-described pen type. The add-on device is adapted to determine the amount of drug expelled from the drug delivery device during an expelling event, i.e. the subcutaneous injection of a dose of drug. In the shown embodiment determination of an expelled dose of drug is based on determination of scale drum position at the beginning and at the end of the expelling event. To determine the rotational position of the scale drum the dose numerals as seen in the display window 102 may be captured and used, this allowing an unmodified pen device to be used. Actual determination of scale drum position may be performed using e.g. optical character recognition (OCR) or template matching. The latter concept is described in greater detail in EP 15202796.7 which is hereby incorporated by reference. Alternatively a dedicated code pattern may be provided on the scale drum as disclosed in e.g. WO 2013/004843.

The add-on device comprises a housing 201 in which are arranged electronic circuitry 210 powered by an energy source 211. The electronic circuitry is connected to and interacts with a light source 220 adapted to illuminate at least a portion of the scale drum 170 seen in the window 102, a capture device (camera device) 221 adapted to capture image data from the scale drum, a low-power motion/position sensor assembly 235 designed to interface with the dose setting member 180 either directly or indirectly, a mounting switch 230 adapted to engage the pen housing 101, a display 240 and user input means in the form of one or more buttons 250. Additionally an acoustic sensor may be provided to detect specific sounds generated by the expelling mechanism during dose setting and dose expelling. The electronic circuitry 210 will typically comprise controller means, e.g. in the form of a generic microprocessor or an ASIC, non-volatile program memory such as a ROM providing storage for embedded program code, writable memory such as flash memory and/or RAM for data, and a display controller. The electronic circuitry may also comprise one or more means of removing or communicating information stored in ROM or flash memory such as a wireless transmitter/receiver, a card slot or an output port, e.g. a USB port.

The add-on device further comprises mounting means (not shown) adapted to releasably mount and securely hold and position the add-on device on the pen housing. As appears the add-on device covers the display window for which reason the current dose size shown in the display window has to be captured and displayed on the electronic display 240.

The coupling means may be in the form of e.g. a bore allowing the add-on device to slide in place on the pen body, flexible gripping structures allowing the add-on device to be mounted in a perpendicular direction, or locking means which has to be operated by the user, e.g. a hinged latch member or a sliding member. In order to securely hold and position the add-on device on the pen housing the add-on device may be provided with positioning means adapted to engage a corresponding positioning structure on the pen body. The positioning structure may be in the form of an existing structure provided for a different purpose, e.g. the window opening, or a specific mounting structure, e.g. one or more indents provided on the pen body.

As scale drum position and thus dose size determination is based on image capturing and subsequent processing of the captured image data, it is important that the add-on device is correctly positioned in its intended operational position on the drug delivery device. In addition to the above-described coupling and positioning means designed to provide a user-recognisable engagement, e.g. by an ensuring “click”, the add-on device 200 is provided with a mounting switch 230, e.g. a mechanical micro switch, which is actuated from an off-state to an on-state when the add-on device is mounted on the pen housing.

As mentioned above, the add-on device is provided with a low-power motion/position sensor assembly 235 which is designed to control the display 240 during dose setting. In this way the relatively power-hungry camera device and light source do not have to be operated during dose setting to control the display but can be reserved to capture information relating to the expelled dose, this providing a longer battery life. The sensor assembly may be adapted to interact directly with the dose setting member, or the sensor assembly may comprise an additional member adapted to non-rotationally engage the pen dose setting member, the sensor per se detecting rotational motion of the additional member. Embodiments of both types of sensor assemblies are described in greater detail in EP application 16171883.8.

The technology used to capture scale drum position at the beginning and end of a dose expelling event in order to determine the size of an expelled dose may also be used during dose setting, the captured data being used to control an electronic display during dose setting, this as disclosed in e.g. WO 2013/120776. To reduce power consumption during dose setting operations the image capturing and processing means may be operated in different modes, e.g. in a “simple” low-power mode to control the display during dose setting and in a “full” high-power mode to securely determine a set dose size when a given dose has been set/expelled and the scale drum does no longer rotate.

As appears, providing a virtual display for an add-on accessory device which covers the scale drum window during operation results in a higher degree of complexity just as the required additional display adds to the size and cost of the device. Further, although it may be possible to provide a virtual display which is accurate and reliable to a very high degree, users may be concerned that this is not always the case.

With reference to FIG. 5 an exemplary embodiment of the present invention will be described addressing these issues.

More specifically, FIG. 5 shows a schematic representation of an add-on device (or module) 300 in a state where it has been mounted on the housing 101 of a drug delivery device 100 of the above-described pen type. The add-on device is adapted to determine the amount of drug expelled from the drug delivery device during an expelling event, i.e. the subcutaneous injection of a dose of drug. In the shown embodiment determination of an expelled dose of drug is based on determination of scale drum position at the beginning and at the end of the expelling event.

The add-on device comprises a housing 301 having a first surface 302 serving as a mounting surface, a capture opening 370 formed in the first surface, and a viewing opening 340 arranged opposed to the capture opening. In the housing is arranged electronic circuitry 310 powered by an energy source 311. The electronic circuitry is connected to and interacts with a light source 320 adapted to illuminate at least a portion of the scale drum 170 seen in the pen window 102, as well as a capture assembly 325 comprising a capture device (camera device) 321 adapted to capture image data from the scale drum. The capture assembly also comprises an optical lens assembly 322 and an optical filter 323. The add-on device further comprises a dichroic beam splitter (see-through mirror) 350 above the scale drum window 102, and a mirror 360 for redirecting light from the beam splitter towards the camera device, whereby it is possible for the camera device to be located away from the scale drum opening and still “see” the scale drum via the “mirror side” of the beam splitter. In the shown embodiment the camera device is positioned in such a way that an additional camera mirror is required. Alternatively the camera mirror may be dispensed with and the camera device may be positioned corresponding to the shown position for the camera mirror. While the camera captures the scale drum, the user can via a transparent housing window 340 see through the beam splitter 350 and thereby directly observe the scale drum during dose setting. By this arrangement both the user and the camera observes the scale drum in an essentially perpendicular viewing direction through the capture opening 370.

The shown embodiment is optimized for allowing the camera device to work in the infrared (IR) light spectrum. More specifically, IR light generated by the IR light source 320 is directed towards the scale drum portion 170 showing in the window opening 102 and reflected therefrom towards the lower surface of the dichroic beam splitter 350 which is optimized for allowing visible light (to the human eye) to pass and to reflect IR light towards the camera mirror 360. The camera assembly is provided with a lens system 323 comprising one or more lenses for focusing the scale drum image on the camera device 321 as well as a light filter. The IR filter of the camera assembly and the light source are “paired” light spectrum wise with the reflective side of the beam splitter. The spectrum can be within the IR spectrum or alternatively outside, but for optimum performance pairing of the camera, lens, light and reflective side of the beam splitter is important.

As for the above-described embodiment of FIG. 4 determination of an expelled dose of drug in the embodiment of FIG. 5 is based on determination of scale drum position at the beginning and at the end of the expelling event.

Depending on the desired level of device complexity, the user interface may be more or less user friendly, i.e. a high degree of user friendliness will normally require a higher level of device complexity and a lower degree of user friendliness will normally require a lower level of device complexity.

In a relatively simple system an optical motion detector system may be based on the above-described capturing system, the captured images being analysed to detect motion of the scale drum, this indicating to the system that a dose is being set. When stop of motion is detected this would indicate that a desired dose has been set which could then be determined based on image analysis of the captured scale drum image. When motion subsequently is determined this would indicate that a dose is being expelled. When stop of motion is detected this would then indicate that a dose has been expelled and the position of the scale drum could then be determined, e.g. zero indicating that a given set dose has been fully expelled, this allowing the size of the expelled dose to be calculated based on the start and end values.

However, such a simple system would come with a number of drawbacks. For example, when a dose has been set and the position of the scale drum has been captured, the user may desire to reduce the set dose. Indeed, this should not be interpreted as an expelling event. Correspondingly, for such a “simple” system the optical sensor would have to be able to determine whether the scale drum is moving during dose setting or during expelling. For example, the motion sensor may be designed to detect the speed of rotation of the scale drum, i.e. relatively slow rotation during dose setting in both directions and relatively fast rotation during dose expelling. In case the user initially would rotate the scale drum to an approximate dose value and then fine-adjust the set dose, such a situation could be identified based on the starting value, i.e. zero. However, as appears, such a system may work well in most situations but would not be perfect.

In the alternative the add-on device may be designed to provide a higher degree of user friendliness and be less focused on low system complexity.

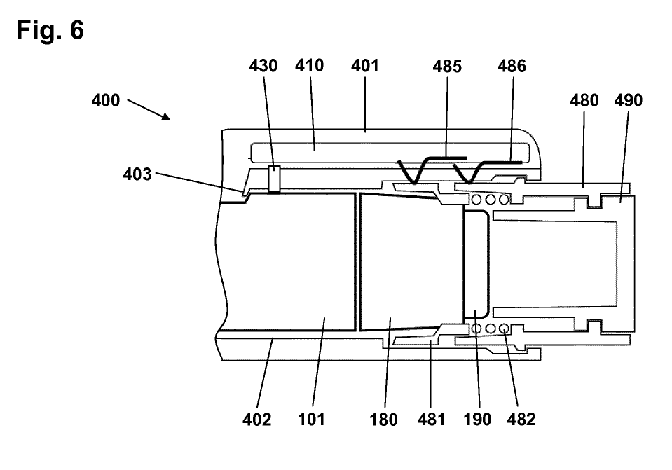

Turning to FIG. 6, a second embodiment of an add-on module 400 is shown in part, the module comprising a state switch actuated by a release member provided as part of the module. More specifically, the add-on module 400 comprises a housing 401 in which are arranged electronic circuitry 410, an energy source, a light source, and a camera device (as in the FIG. 5 embodiment). The add-on module housing forms a bore adapted to receive the generally cylindrical proximal housing portion 101 of the pen device, the bore being defined by a generally cylindrical mounting surface 402 adapted to face the pen device. A viewing/camera opening is formed in the mounting surface. A small clearance is provided between the bore and the pen to allow the pen to be received in the bore. A firm grip between the two structures is provided by a locking structure on the add-on module adapted to engage the pen device and securing a firm grip. The locking structure may be spring biased and adapted to snap in place when the add-on module is mounted on the pen. Alternatively the locking structure may be in the form of a user-operated lock. The add-on module comprises a positioning structure 403 protruding from the mounting surface and being adapted to engage at least portions of the window opening edge to thereby position the add-on module both axially and rotationally relative to the opening, e.g. in the form of a protruding lip 403 structure surrounding at least partially the viewing/camera opening, the lip structure being adapted to formfitting engage the edge portion of the window opening to thereby ensure correct positioning both axially and rotationally between the two housing structures and thereby between the camera device and the scale drum.

Additionally, the add-on module 400 is provided with a user accessible add-on dial member 480, a user accessible add-on release button 490, a motion sensor assembly comprising a cylindrical motion member 481 in combination with a motion detector 485, and a state switch (or sensor) 486 associated with the add-on dial member. In the shown embodiment the add-on dial member 480 and the add-on release button 490 is mounted axially locked to each other to form a combined add-on dial and release member. The two members may also be rotationally locked to each other. As appears, when the add-on module is mounted on the pen device the pen dose setting member and the pen release button are covered by the add-on module.

The add-on module further comprises a mounting switch assembly 430 arranged in the vicinity of the positioning structure 403. The switch assembly 430 is in communication with the electronic circuitry and may comprise an actuator element arranged to move between a biased distal “off” position in which it protrudes into the bore and a retracted proximal “on” position, the latter indicating to the electronic circuitry that the add-on module has been mounted on a pen corresponding pen body.

In respect of the working principle of the FIG. 6 embodiment, the cylindrical motion member 481 has an inner surface adapted to axially engage the dose setting member 180 when the add-on device is mounted on the pen device, the mating surfaces providing non-rotational engagement, e.g. based on the axially oriented groove pattern provided on the FlexTouch® pen device as shown in FIG. 1A. The add-on dial member 480 is coupled freely rotatable to the module housing 401 but is in non-rotational engagement with the motion member 481 and thus the dose setting member 180 when the add-on module is mounted on the pen device. The add-on dial member 480 is arranged to be moved axially relative to the module housing between an initial proximal-most position and an actuated distal-most position. A bias spring 482 is provided between the motion member 481 and the add-on dial member 480 to ensure that the combined add-on member is biased towards its proximal-most position. The add-on release button portion 490 of the combined add-on member is adapted to engage the pen device release button 190 when moved axially relative to the module housing. In this way a dose is set by means of the add-on dial member portion 480 and a set dose is released by means of the add-on release button portion 490.

In addition to the motion sensing feature the motion sensor assembly also provides a wake-up switch arrangement having a low-power sleep mode yet provides a wake-up signal when the motion member is rotated during initial dose setting. As the motion member 481 inevitably will move during mounting of the add-on module, the motion sensor arrangement may also provide a mounting switch which will wake-up the electronics during mounting.

In the shown embodiment the state switch assembly comprises one or more contact members 486 in sliding engagement with the distal portion of the add-on dial member 480 on which a circumferential code ring is arranged, this providing a switch arranged to detect axial movement of the add-on release button 490 between a proximal dose setting state and a distal dose release state.

The motion and state sensors may be based on a mechanical, optical, magnetic or conductive interface with a moving member. For a mechanical design the contact members of both the motion sensor and the state switch may be in the form of flexible contact arms which may be provided in the form a combined contact arm array.

In addition to the state sensing feature the state switch arrangement may also provide a wake-up switch arrangement having a low-power sleep mode yet provides a wake-up signal when the add-on release button is actuated. This arrangement may be relevant during certain operational conditions as will be described below.

Before turning to the description of different use scenarios a further feature of the exemplary system will be described. More specifically, the add-module 400 is provided with a memory in which the last rotational position of the scale drum determined by use of the camera is stored. When the add-on module is mounted on a new pen device the memory has to be reset, however, to avoid that the user will have to perform certain operations as part of the initial mounting procedure, the add-on module may be provided with an automatic scale drum position capture. More specifically, when it is detected by the mounting switch means that the add-on module has been mounted anew (or for the first time) on a pen device, the camera will capture an image of the scale drum which will be processed to determine the position of the scale drum, e.g. by template matching, and stored in the “last-position” memory.

During normal use the user will start dialing a dose which will wake up the system. The stored value will typically, but not necessarily, be zero. As an “absolute” position is determined during dose setting, the system will also be able to handle situations in which a set dose is adjusted, i.e. decreased.

When the system detects that motion has stopped the camera will be operated to capture an image which will be processed to determine the position of the scale drum and the memory will be updated with a new last-position value. The user may desire to further adjust the dose which will result in a new image being captured when motion is detected to have stopped.

At this point the system is awake and waiting for the set dose to be released, however, after e.g. 5 minutes without activity the system may return to sleep mode. To wake up the system when the set dose is released by actuating the add-on release button, input from the state switch may be utilized. As the dose setting member and thus the motion sensor is not moving during out-dosing there will be no motion activity input to wake up the system. Alternatively the system may first wake up when the add-on release button returns to the proximal dose setting position.

When the user after or during dose expelling releases the add-on release button the state switch will detect the change and control the camera device to capture an image of the scale drum. If the scale drum position is determined to be at the end-of-dose zero position it can be assumed that the set dose has been fully expelled and that the size of the expelled dose correspond to the last-position value stored in memory. The data will be stored as a log entry in the module memory. If the camera-captured scale drum position does not correspond to the end-of-dose position the system will at first assume that expelling of the set dose has been paused and await further activity. If no activity is detected the system will after a given amount of time, e.g. 5 minutes, time out and calculate an expelled dose size as the difference between the set dose amount (corresponding to the last-position value) and the remaining dose amount (corresponding to the present-position value). The calculated dose size will stored as a log entry. The previous last-position value will be over-written with the present-position value.

If the user at this point decides to expel the remaining dose, the system will wake up and determine a further expelled dose amount, e.g. corresponding fully or partly to the remaining dose amount. Alternatively, the user may decide to cancel the remaining dose fully or partly by dialing the add-on dial member towards the initial zero position. When it is detected that rotational motion has stopped the camera will be operated to capture an image. As the state switch has not been operated the new captured position value, e.g. zero, will be stored in memory as a new last-position.

In the embodiments of FIGS. 5 and 6 the add-on device does not comprise a display, however, if desired the add-on device may be provided with an electronically controlled display adapted to display data related to determined dose amounts, e.g. the size and the corresponding time for stored dose events. Alternatively or in addition, the add-on device may comprise communication means allowing data related to determined dose amounts to be transferred to an external device, e.g. by wireless transmission means such as NFC or Bluetooth® allowing data to be transferred to and displayed on a smartphone.

In addition to provide input during operation of the pen device, the above-described state switch may also be used to control the add-on module in other ways, e.g. to control a provided display to show stored log entries and/or to control communication between the add-on device and an external device.

As appears, by the sensor set-up of the FIG. 6 embodiment an add-on logging device is provided which is user-friendly to a high degree.

In the above-described add-on devices scale drum position may be determined by template-matching with a stored representation of the entire scale drum surface image.

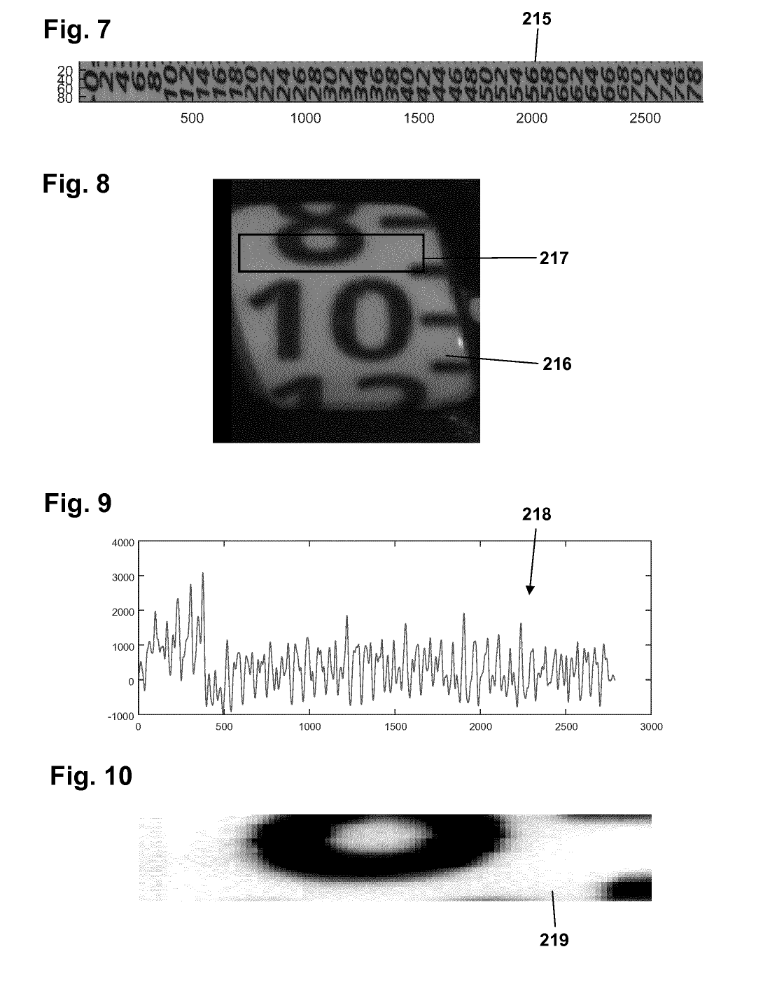

Correspondingly, FIG. 7 illustrates a template image 215 of the whole scale-drum, obtained by concatenating parts of successive images from a film where the scale-drum moves from position 80 to position 0. The template image is used as a reference when to determine the position of a specific image. The pixel position (horizontal axis in the above figure) corresponds to the drum position (in degrees, IU or other units). As an example, FIG. 8 shows an image 216 of the scale-drum window where the position corresponds to 10 IU, the rectangle 217 illustrating the area that is used for position detection. FIG. 9 then shows the cross correlation of the rectangle image portion to the reference 115 as a function 118 of pixel position. Searching for the peak reveals a best match at pixel position 341, corresponding to the cut 119 from the reference image as shown in FIG. 10. The reference image at this pixel off-set was taken when the scale drum was in a position 9.8 IU.

To speed up the search procedure to save time and enhance power efficiency the search may advantageously begin at the position representing the most likely value and, at least initially, only consider a given limited range around that position. Thus the electronic circuitry of the FIG. 6 embodiment is provided with a memory adapted to store a reference position value corresponding to the most recent known rotational position of the indicator, and the processor means is adapted to (i) determine a current rotational position of the indicator based on:

a captured image of the indicator, and the stored reference position value, (ii) store the determined current rotational position in the memory as a new reference position value, and (iii) determine an expelled dose amount based on inputs from the camera as described above. Although the motion detector 485 is not used to control a display, it may optionally be provided with the ability to detect position, the detected position being used to optimize the search procedure. Indeed, such a system could be designed to meet lower standards of precision and reliability as the output would only be used to optimize internal processes in the add-on device. Also in case rotational position of the indicator is based on OCR it may help improve efficiency and accuracy of the OCR process if information of the most likely rotational position is known.

In the above description of exemplary embodiments the image capture device has been described in the form of an add-on device adapted to capture dose data from a drug delivery device to which is has been mounted, however, this is only an exemplary embodiment. For example, the capture device may be in the form of an inspection and documentation device used in e.g. quality control. For such an implementation the viewing opening may be provided with a magnifier allowing the user to study an observed object in detail, the capturing and memory means providing at the same time documentation for the observed object for subsequent retrieval or transmission to an external device.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An image capture device, comprising:
a housing comprising:
a first surface,
a capture opening formed in the first surface, and
a viewing opening arranged opposed to the capture opening,
a memory,
capturing structure adapted to capture an image of at least a portion of an object arranged in register with the capture opening, the capturing structure being arranged off-set in respect of the viewing axis between the viewing opening and the capture opening, and
a processor adapted to process captured images and store data in the memory,
wherein the capture device further comprises a beam-splitter device arranged to cover at least a portion of the capture opening viewable through the viewing opening, the beam-splitter device being adapted to:
allow a first portion of light reflected from an object arranged in register with the capture opening to pass through the beam-splitter device, this allowing a user to watch a portion of the object covered by the beam-splitter device, and
allow a second portion of light reflected from the indicator member to be reflected by the beam-splitter device towards the capturing structure.

2. An image capture device as in claim 1, wherein the capturing structure comprises an image sensor.

3. An image capture device as in claim 1, further comprising a mirror arranged to direct light received from the beam-splitter device towards the capturing structure.

4. An image capture device as in claim 1, further comprising a light source arranged to illuminate at least a portion of an object arranged in the vicinity of the capture opening and viewable through the viewing opening, the capturing structure comprising a light filter adapted to filter out at least a portion of light outside the range of light from the light source.

5. An image capture device as in claim 4, wherein the light source is an IR light source and the light filter is adapted to filter out at least a portion of light outside the IR range.

6. An image capture device as in claim 1, wherein the beam-splitter device is of the dichroic type and adapted to primarily reflect IR light and transmit visible light.

7. An image capture device as in claim 1, wherein the processor is adapted to:
recognize a numerical value in a captured image,
determine and store in the memory a calculated numeric value representing the difference between first and second recognized numerical values.

8. An image capture device as in claim 1 configured to be releasably attached to a drug delivery device, the drug delivery device comprising:
a drug reservoir or a compartment for receiving a drug reservoir,
drug expelling structure comprising a dose setting member allowing a user to set a dose amount of drug to be expelled,
an indicator member adapted to rotate relative to the housing during dose setting and dose expelling corresponding to an axis of rotation, the amount of rotation corresponding to a set dose respectively the amount of drug remaining to be expelled from a reservoir by the expelling structure, the indicator member having an initial rotational position corresponding to no dose amount being set,
a housing comprising an opening allowing a user to observe a portion of the indicator member, and
a pattern arranged circumferentially or helically on the indicator member, comprising a plurality of indicia, the currently observable indicia indicating to a user the size of a currently set dose amount of drug to be expelled,
the image capture device being adapted to determine, when mounted to a drug delivery device housing, an expelled dose amount, the image capture device comprising:
mounting structure adapted to releasably mount the image capture device to the drug delivery device in a predetermined position and orientation with the first surface facing the drug delivery housing and the capture opening arranged in register with the housing opening,
wherein the capturing structure is adapted to capture an image of at least a portion of a currently observable indicia on the indicator member, and
wherein the processor is adapted to determine and store an expelled dose-amount based on first and second captured images of the indicator member, the first image being captured when a dose amount has been set and the second image being captured when a dose amount has been expelled.

9. An image capture device as in any claim 8, further comprising motion sensor structure adapted to detect that a dose is being set.

10. An image capture device as in claim 9, wherein the motion sensor structure is adapted to detect motion based on images captured by the capturing structure and thereby motion of the indicator member.

11. An image capture device as in claim 10, wherein the motion sensor structure is adapted to detect motion of the dose setting member.

12. An image capture device as in claim 9, wherein the processor is adapted to determine expelled dose amounts based in part on motion detected by the motion sensor structure.

13. An image capture device as in claim 8, comprising:
a release member actuatable between a dose setting state and an expelling state in which the drug expelling structure is released to expel a set dose, and
state sensor structure adapted to detect the state of the release member.

14. An image capture device as in claim 8, comprising at least one of (i) a display adapted to display data related to determined dose amounts, and (ii) communication structure allowing data related to determined dose amounts to be transferred to an external device.

15. An image capture device as in claim 8 in combination with a drug delivery device wherein the processor is adapted to:
recognize a numerical value in a captured image,
determine and store in the memory a calculated numeric value representing the difference between first and second recognized numerical values.
